(19) United States
(12) Patent Application Publication
(10) Pub. No.: US 2002/0107196 A1
(43) Pub. Date: Aug. 8, 2002

(54) Method for inducing chemotaxis in endothelial cells by administering stromal cell derived factor-1alpha

(75) Inventor: Shalley K. Gupta, Lafeyette Hill, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/953,717

(22) Filed: Sep. 17, 2001

Related U.S. Application Data
(62) Division of application No. 09/358,624, filed on Jul. 21, 1999.
(60) Provisional application No. 60/093,596, filed on Jul. 21, 1998.

(51) Int. Cl. A61K 38/17

(52) U.S. Cl. 514/12

(57) ABSTRACT

CXCR4 and SDF-1α polypeptides and polynucleotides and methods for producing such polypeptides by recombinant techniques are disclosed. Also disclosed are methods for utilizing CXCR4 and SDF-1α polypeptides and polynucleotides in the design of protocols for the treatment of inflammatory diseases, angiogenic diseases, and infections, such Human Immunodeficiency Virus (HIV).

### CROSS-REFERENCE TO RELATED APPLICATION

This application claims benefit to the earlier provisional U.S. Application No. 60/093,596, filed on Jul. 21, 1998, the contents of which are incorporated herein by reference in their entirety.

### FIELD OF INVENTION

This invention relates to methods for inducing the chemotaxis of endothelial cells in a patient, thereby stimulating angiogenesis in the vasculature of the patient, comprising contacting the endothelial cells with a chemotaxis-inducing-effective amount of stromal cell derived factor-la (herein “SDF-1α”). Also contemplated within the scope of the invention are methods of inhibiting or stimulating angiogenesis in the vasculature of a patient comprising contacting the vasculature with either an angiogenesis-stimulating-effective amount of an agonist or an angiogenesis-inhibiting-effective antagonist of the interaction between of CXCR4 and SDF1-α.

### BACKGROUND OF THE INVENTION

The CXCR-4 receptor, originally known as LESTR/fusin, was shown to be the receptor for the α or CXC chemokine, stromal cell-derived factor-1 (SDF-1) in 1996 (Feng, et al., Science 272: 872-877 (1996)) and renamed at this time. SDF-1αis specific for CXCR-4 and mediates its chemotactic effects via this receptor. The CXCR4 receptor is widely expressed in a variety of cell types and is implicated in a range of inflammatory responses mediated by SDF-1α. In addition, CXCR-4 has been identified as the co-receptor used by T-tropic HIV-1 viral strains to infect cells, and antagonists of the receptor would, therefore, be useful in the treatment of late stage HIV infection and AIDS.

The vascular response to infection and inflammation is characterized by the adhesion of leukocytes to the endothelium, and their transmigration from the circulation at sites of tissue injury. Along with EC, chemokines play an active role in this complex process by defining the types of leukocytes that get recruited in response to the inflammatory stimuli. Ben-Baruch, et al., J. Biol. Chem. 270, 11703-11706 (1995). Previous studies have suggested that for chemokines to efficiently mediate their chemotactic role, would require the presence of binding sites on the EC surface. Rot, A., Immunol. Today 13: 291-294 (1992); Tanaka, et al., Immunol. Today 14: 111-114 (1993); Rot, et al., J. Leuk. Biol. 59: 39-44 (1996). Although chemokines can strongly bind to cell surface proteoglycans (Dragic, et al., Nature 381: 667-673 (1996)), there is little molecular evidence to support EC expression of specific chemokine receptors. Shaw, et al., Cell 46: 659-667 (1986).

Chemokines play an important role in the regulation of endothelial cell (EC) function, including proliferation, migration and differentiation during angiogenesis and re-endothelialization after injury. See Gupta, et al., JBC 273(7): 4282-4287 (1997). Expression of CXCR4 in EC is significant, as it and several CC chemokine receptors are thought to serve as fusion co-factors along with CD4 during HIV infection. The studies also lend support to reports of EC susceptibility to HIV infection in a CD4-independent manner. Taken together, these findings provide evidence of chemokine receptor expression in EC, elucidate the action of SDF-1β on the vascular endothelium. The vascular endothelium is strategically located to play a prominent sensory and effector cell role in the maintenance of hemostasis, and during the vascular response to inflammation, infection and injury. Pober, et al., Transplantation 50: 537-544 (1990); Mantovani, et al., FASEB J. 6: 2591-2599 (1992). The endothelium is also integrally associated with angiogenesis (Maciag, T. & Burgess, W. H., ENDOTHELIAL CELLS, Vol II eds. Ryan, U.S. (CRC Press, Inc., Boca Raton, Fla.), pp. 3-10 (1988)) and cardiovascular disorders, such as atherosclerosis and restenosis. Gibbons, et al., N. Engl. J. Med. 330: 1431-1438 (1994). Endothelial cells (EC) interact with various inflammatory cells, as well as platelets and smooth muscle cells via a variety of chemotactic factors such as chemokines and their receptors. Ben-Baruch, et al., J. Biol. Chem. 270: 11703-11706 (1995); Strieter, et al., Science 243(4897):1467-1469 (1989).

The Applicant has found that, due to EC expression of CXCR4, its ligand, SDF-1β plays a direct role in angiogenesis, thereby modulating the vascular endothelium responses to inflammation, injury, ischaemia, and infections, such as Human Immunodeficiency Virus (HIV). Clearly there is a need for identification and characterization of further agonists and antagonists of the interaction between CXCR4 and SDF-1β that play a role in preventing, ameliorating or correcting dysfunctions or diseases, including, but not limited to: viral (i.e., Acquired Immunodeficiency Syndrome (AIDS)), bacterial, fungal and protozoan infections, pain, cancer, diabetes, obesity, anorexia, bulimia, asthma, allergies, Parkinson's disease, acute heart failure, hypotension, hypertension, urinary retention, osteoporosis, angina pectoris, myocardial infarction, stroke, ulcers, benign prostatic hypertrophy, migraine, vomiting, psychotic and neurological disorders (i.e., anxiety, schizophrenia, manic depression, depression, delirium, dementia, mental retardation, etc.) and dyskinesias (i.e., Huntington's disease and Gilles de la Tourette's syndrome, etc.), inflammatory diseases, such as rheumatoid arthritis, diabetic retinopathy, inflammatory bowel disease, atherosclerosis, restenosis, stroke, Alzheimer's disease, congestive heart failure, and cardiac remodeling; angiogenic diseases, such as solid tumors (i.e., colon cancer, esophageal cancer, breast cancer, etc.), Kaposi Sarcoma, rheumatoid arthritis, diabetic retinopathy; and spinal cord injury, among others.

### SUMMARY OF THE INVENTION

In one aspect, the invention relates to a method for inducing chemotaxis in endothelial cells comprising contacting the cells with a chemotaxis inducing effective amount of stromal cell derived factor-1α (SDF 1-α) (SEQ ID NO:4) in combination with a carrier.

In another aspect, the invention relates to a method for stimulating angiogenesis in the vasculature of a patient in need thereof comprising contacting the vasculature with an angiogenesis-stimulating-effective amount of SDF 1-α (SEQ ID NO:4) in combination with a carrier.

Yet another aspect of the invention relates to a method for stimulating angiogenesis in the vasculature of a patient comprising contacting the vasculature with an angiogenesis-stimulating-effective amount of an agonist of the interaction between CXCR4 (SEQ ID NO:2) and SDF 1-α (SEQ ID NO:4), wherein the patient is suffering from atherosclerosis, restenosis, iscchaemic stroke, and spinal cord injury.

In another aspect, the invention relates to a method for inhibiting angiogenesis in the vasculature of a patient comprising contacting the vasculature with an angiogenesis-inhibiting-effective amount of an antagonist of the interaction between CXCR4 (SEQ ID NO:2) and SDF 1-α (SEQ ID NO:4), wherein the patient is suffering from a disease or disorder including, but not limited to: viral (i.e., Acquired Immunodeficiency Syndrome (AIDS)), bacterial, fungal and protozoan infections, pain, cancer, diabetes, obesity, anorexia, bulimia, asthma, allergies, Parkinson's disease, acute heart failure, hypotension, hypertension, urinary retention, osteoporosis, angina pectoris, myocardial infarction, stroke, ulcers, benign prostatic hypertrophy, migraine, vomiting, psychotic and neurological disorders (i.e., anxiety, schizophrenia, manic depression, depression, delirium, dementia, mental retardation, etc.) and dyskinesias (i.e., Huntington's disease and Gilles de la Tourette's syndrome, etc.), inflammatory diseases, such as rheumatoid arthritis, diabetic retinopathy, inflammatory bowel disease, atherosclerosis, restenosis, stroke, Alzheimer's disease, congestive heart failure, and cardiac remodeling; angiogenic diseases, such as solid tumors (i.e., colon cancer, esophageal cancer, breast cancer, etc.), Kaposi Sarcoma, rheumatoid arthritis, diabetic retinopathy.

### BRIEF DESCRIPTION OF THE DRAWINGS

FIG. 1 shows the nucleotide and deduced amino acid sequence from human CXCR4 (SEQ ID NOs:1 and 2).

FIG. 2 shows the nucleotide and deduced amino acid sequence from human SDF-1α (SEQ ID NOs:3 and 4).

### DESCRIPTION OF THE INVENTION

Definitions

The following definitions are provided to facilitate understanding of certain terms used frequently herein.

“CXCR4” refers, among others, generally to a polypeptide having the amino acid sequence set forth in SEQ ID NO:2 or an allelic variant thereof.

“SDF1-α” refers, among others, generally to a polypeptide having the amino acid sequence set forth in SEQ ID NO:4 or an allelic variant thereof.

“CXCR4 activity” or “CXCR4 polypeptide activity” or “biological activity of the CXCR4 receptor or CXCR4 polypeptide” refers to the metabolic or physiologic function of human CXCR4, including similar activities or improved activities or these activities with decreased undesirable side-effects.

“SDF1-α activity” or “SDF1-α polypeptide activity” or “biological activity of SDF1-α or SDF1-α polypeptide” refers to the metabolic or physiologic function of human SDF1-α, including similar activities or improved activities or these activities with decreased undesirable side-effects.

“CXCR4 gene” refers to a polynucleotide having the nucleotide sequence set forth in SEQ ID NO:1 or allelic variants thereof and/or their complements.

“SDF1-α gene” refers to a polynucleotide having the nucleotide sequence set forth in SEQ ID NO:3 or allelic variants thereof and/or their complements.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol. (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Altschul, et al., J. Molec. Biol. 215: 403-410 (1990). The BLAST X program is publicly available from NCBI and other sources (BLAST Manual, Altschul, et al., NCBI NLM NIH Bethesda, Md. 20894; Altschul, S., et al., J. Mol. Biol. 215: 403-410 (1990). The well known Smith Waterman algorithm may also be used to determine identity.

Preferred parameters for polypeptide sequence comparison include the following:

1) Algorithm: Needleman, et al., J. Mol Biol. 48: 443-453 (1970)

Comparison matrix: BLOSSUM62 from Hentikoff, et al., Proc. Natl. Acad. Sci. USA. 89:10915-10919 (1992).

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman, et al., J. Mol Biol. 48: 443-453 (1970).

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity(divided by 100) and subtracting that product from said total number of nucleotides in SEQ ID NO:1, or:

nₙ≦xₙ−(xₙ·y)

wherein nₙ is the number of nucleotide alterations, xₙ is the total number of nucleotides in SEQ ID NO:1, and y is, for instance, 0.70 for 70%, 0.80 for 80%, 0.85 for 85%, 0.90 for 90%, 0.95 for 95%, etc., and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity(divided by 100) and then subtracting that product from said total number of amino acids in SEQ ID NO:2, or:

nₐ≦xₐ−(xₐ·y),

wherein nₐ is the number of amino acid alterations, xₐ is the total number of amino acids in SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ.

Polypeptides of the Invention

In one aspect, the present invention relates to CXCR4 polypeptides. The CXCR4 polypeptides include the polypeptide of SEQ ID NO:2; as well as polypeptides comprising the amino acid sequence of SEQ ID NO:2; and polypeptides comprising an amino acid sequence having at least a 95% identity to that of SEQ ID NO:2 over its entire length. Preferably CXCR4 polypeptides exhibit at least one biological activity of human CXCR4.

In another aspect, the present invention relates to SDF 1-α polypeptides. The SDF1-α polypeptides include the polypeptide of SEQ ID NO:4; as well as polypeptides comprising the amino acid sequence of SEQ ID NO:4; and polypeptides comprising an amino acid sequence having at least a 95% identity to that of SEQ ID NO:4 over its entire length. Preferably SDF1-α polypeptides exhibit at least one biological activity of human SDF1-α.

The CXCR4 and SDF1-α polypeptides may be in the form of the “mature” protein or may be a part of a larger protein such as a fusion protein. It is often advantageous to include an additional amino acid sequence that contains secretory or leader sequences, pro-sequences, sequences that aid in purification such as multiple histidine residues, or an additional sequence for stability during recombinant production.

Biologically active fragments of the CXCR4 and SDF1-α polypeptides are also included in the invention. A fragment is a polypeptide having an amino acid sequence that entirely is the same as part, but not all, of the amino acid sequence of the aforementioned CXCR4 and SDF 1-α polypeptides. As with CXCR4 and SDF 1-α polypeptides, fragments may be “free-standing,” or comprised within a larger polypeptide of which they form a part or region, most preferably as a single continuous region. Representative examples of polypeptide fragments of the invention, include, for example, fragments from about amino acid number 1-20, 21-40, 41-60, 61-80, 81-100, and 101 to the end of the human CXCR4 and SDF1-α polypeptides. In this context, “about” includes the particularly recited ranges larger or smaller by several, 5, 4, 3, 2 or 1 amino acid at either extreme or at both extremes.

Preferred fragments include, for example, truncation polypeptides having the amino acid sequence of CXCR4 and SDF1-α polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Biologically active fragments are those that mediate CXCR4 or SDF1-α activity, including those with a similar activity or an improved activity, or with a decreased undesirable activity. Also included are those that are antigenic or immunogenic in an animal, especially in a human.

Preferably, all of these polypeptide fragments retain the biological activity of CXCR4 or SDF 1-α, including antigenic activity. Variants of the defined sequence and fragments also form part of the present invention. Preferred variants are those that vary from the referents by conservative amino acid substitutions—Le., those that substitute a residue with another of like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

The CXCR4 and SDF1-α polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Polynucleotides of the Invention

Another aspect of the invention relates to CXCR4 polynucleotides. CXCR4 polynucleotides include isolated polynucleotides encoding the CXCR4 polypeptides and fragments, and polynucleotides closely related thereto. More specifically, the CXCR4 polynucleotides of the invention include a polynucleotide comprising the nucleotide sequence set forth in SEQ ID NO:1 encoding a human CXCR4 polypeptide of SEQ ID NO:2, and a polynucleotide having the particular sequence of SEQ ID NO:1. CXCR4 polynucleotides further include a polynucleotide comprising a nucleotide sequence having at least a 95% identity to a nucleotide sequence encoding the human CXCR4 polypeptide of SEQ ID NO:2 over its entire length, and a polynucleotide having at least a 95% identity SEQ ID NO:1 over its entire length. In this regard, polynucleotides at least 97% identical are particularly preferred, and those with at least 98-99% are most highly preferred, with at least 99% being the most preferred. Also included under CXCR4 polynucleotides are nucleotide sequences having sufficient identity to a nucleotide sequence contained in SEQ ID NO:1 to hybridize under conditions useable for amplification or for use as a probe or marker. The invention also provides polynucleotides that are complementary to such CXCR4 polynucleotides.

Another aspect of the invention relates to SDF1-α polynucleotides. SDF1-α polynucleotides include isolated polynucleotides encoding the SDF1-α polypeptides and fragments, and polynucleotides closely related thereto. More specifically, the SDF 1-α polynucleotides of the invention include a polynucleotide comprising the nucleotide sequence set forth in SEQ ID NO:3 encoding a human SDF1-α polypeptide of SEQ ID NO:4, and a polynucleotide having the particular sequence of SEQ ID NO:3. SDF1-α polynucleotides further include a polynucleotide comprising a nucleotide sequence having at least a 95% identity to a nucleotide sequence encoding the human SDF1-α polypeptide of SEQ ID NO:4 over its entire length, and a polynucleotide having at least a 95% identity to SEQ ID NO:3 over its entire length. In this regard, polynucleotides at least 97% identical are particularly preferred, and those with at least 98-99% are most highly preferred, with at least 99% being the most preferred. Also included under SDF1-α polynucleotides are nucleotide sequences having sufficient identity to a nucleotide sequence contained in SEQ ID NO:3 to hybridize under conditions useable for amplification or for use as a probe or marker. The invention also provides polynucleotides that are complementary to such CXCR4 polynucleotides.

The polynucleotides of the present invention encoding CXCR4 and SDF-1α may be obtained using standard cloning and screening, from a cDNA library derived from mRNA in cells of human endothelial cells, peripheral blood leukocytes, spleen, thymus, brain, lung, heart, placenta, etc., using the expressed sequence tag (EST) analysis (Adams, et al. Science 252:1651-1656 (1991); Adams, et al., Nature, 355:632-634 (1992); Adams, et al., Nature 377 Supp:3-174 (1995)). Polynucleotides of the invention can also be obtained from natural sources such as genomic DNA libraries or can be synthesized using well known and commercially available techniques.

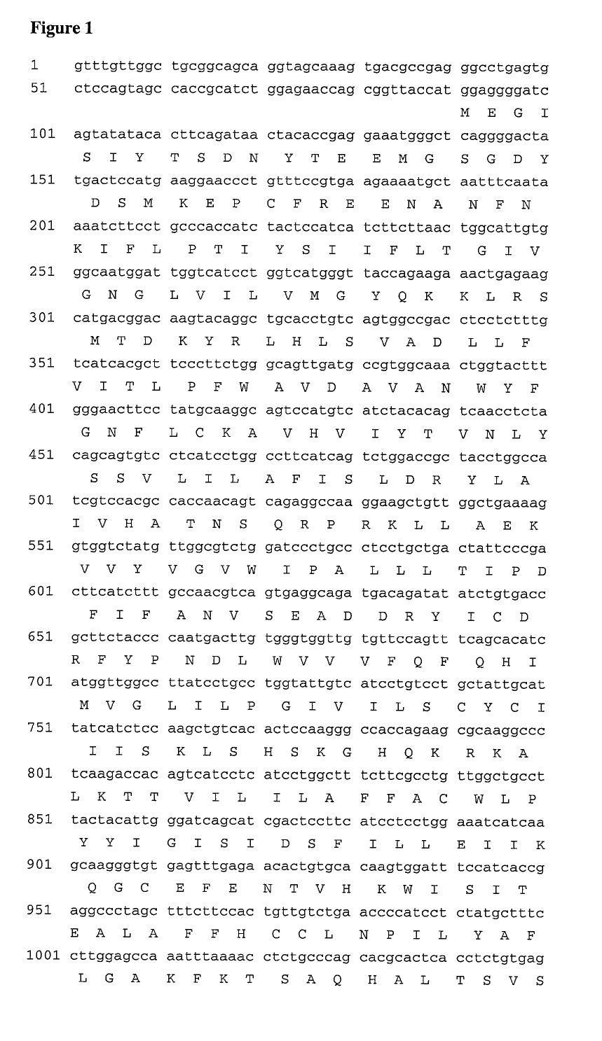

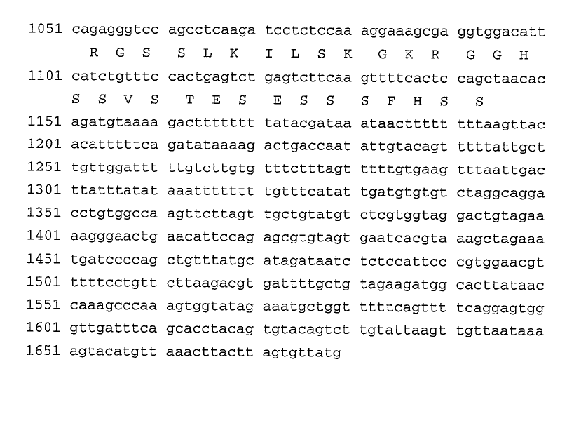

The nucleotide sequence encoding CXCR4 polypeptide of SEQ ID NO:2 may be identical over its entire length to the coding sequence set forth in FIG. 1 (SEQ ID NO:1), or may be a degenerate form of this nucleotide sequence encoding the polypeptide of SEQ ID NO:2, or may be highly identical to a nucleotide sequence that encodes the polypeptide of SEQ ID NO:2. Preferably, the polynucleotides of the invention comprise a nucleotide sequence that is highly identical, at least 95% identical, with a nucleotide sequence encoding a CXCR4 polypeptide, or at least 95% identical with the polynucleotide sequence contained in FIG. 1 (SEQ ID NO:1) encoding CXCR4 polypeptide, or at least 95% identical to a nucleotide sequence encoding the polypeptide of SEQ ID NO:2.

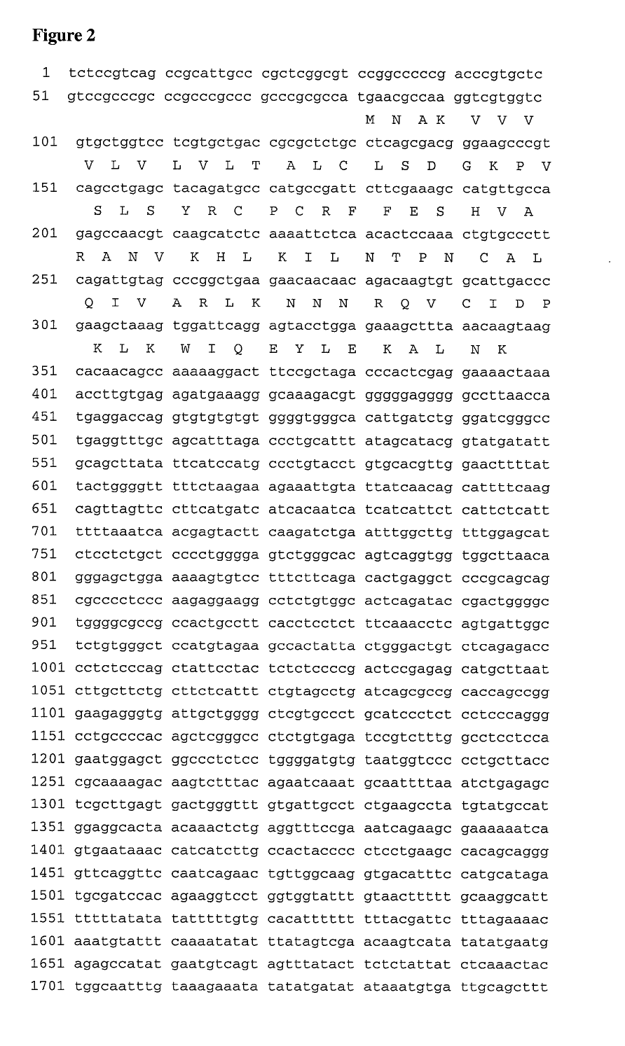

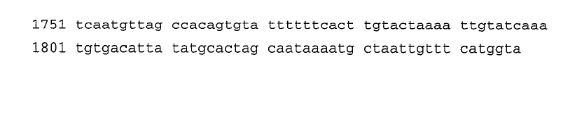

The nucleotide sequence encoding SDF-1α polypeptide of SEQ ID NO:4 may be identical over its entire length to the coding sequence set forth in FIG. 2 (SEQ ID NO:3), or may be a degenerate form of this nucleotide sequence encoding the polypeptide of SEQ ID NO:4, or may be highly identical to a nucleotide sequence that encodes the polypeptide of SEQ ID NO:4. Preferably, the polynucleotides of the invention comprise a nucleotide sequence that is highly identical, at least 95% identical, with a nucleotide sequence encoding SDF-1α polypeptide, or at least 95% identical with the polynucleotide sequence contained in FIG. 2 (SEQ ID NO:3) encoding SDF-1α polypeptide, or at least 95% identical to a nucleotide sequence encoding the polypeptide of SEQ ID NO:4.

When the polynucleotides of the invention are used for the recombinant production of CXCR4 and SDF-1α polypeptide, the polynucleotide may include the coding sequence for the mature polypeptide or a fragment thereof, by itself; the coding sequence for the mature polypeptide or fragment in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz, et al., Proc Natl Acad Sci USA 86:821-824 (1989), or is an HA tag. The polynucleotide may also contain non-coding 5′ and 3′ sequences, such as transcribed, non-translated sequences, splicing and polyadenylation signals, ribosome binding sites and sequences that stabilize mRNA.

Further preferred embodiments are polynucleotides encoding CXCR4 variants that comprise the amino acid sequence of CXCR4 polypeptide of FIG. 1 (SEQ ID NO:2) in which several, 5-10, 1-5, 1-3, 1-2 or 1 amino acid residues are substituted, deleted or added, in any combination. Still further preferred embodiments are polynucleotides encoding SDF-1α variants that comprise the amino acid sequence of SDF-1α polypeptide of FIG. 2 (SEQ ID NO:4) in which several, 5-10, 1-5, 1-3, 1-2 or 1 amino acid residues are substituted, deleted or added, in any combination.

The present invention further relates to polynucleotides that hybridize to the herein above-described sequences. In this regard, the present invention especially relates to polynucleotides that hybridize under stringent conditions to the herein above-described polynucleotides. As herein used, the term “stringent conditions” means hybridization will occur only if there is at least 95% and preferably at least 97% identity between the sequences.

Polynucleotides of the invention, which are identical or sufficiently identical to the nucleotide sequences contained in SEQ ID NO:1 or 3, may be used as hybridization probes for cDNA and genomic DNA, to isolate full-length cDNAs and genomic clones encoding CXCR4 or SDF-1α polypeptides and to isolate cDNA and genomic clones of other genes that have a high sequence similarity to the CXCR4 and SDF-1α genes. Such hybridization techniques are known to those of skill in the art. Typically these nucleotide sequences are at least 95% identical to that of the referent. The probes generally will comprise at least 15 nucleotides. Preferably, such probes will have at least 30 nucleotides and may have at least 50 nucleotides. Particularly preferred probes will range between 30 and 50 nucleotides.

In one embodiment, obtaining a polynucleotide encoding CXCR4 or SDF-1α comprises the steps of screening an appropriate library under stringent hybridization conditions with a labeled probe having the nucleotide sequence of SEQ ID NO:1 or 3 or a fragment thereof; and isolating full-length cDNA and genomic clones containing said polynucleotide sequence. Such hybridization techniques are well known to those of skill in the art. Stringent hybridization conditions are as defined above or alternatively conditions under overnight incubation at 42° C. in a solution comprising: 50% formamide, 5× SSC (150 mM NaCl, 15 mM trisodium citrate), 50 mM sodium phosphate (pH 7.6), 5× Denhardt's solution, 10% dextran sulfate, and 20 microgram/ml denatured, sheared salmon sperm DNA, followed by washing the filters in 0.1× SSC at about 65° C.

The polynucleotides and polypeptides of the present invention may be employed as research reagents and materials for discovery of treatments and diagnostics to animal and human disease.

Vectors, Host Cells, Expression

The present invention also relates to vectors that comprise a polynucleotide or polynucleotides of the present invention, and host cells that are genetically engineered with vectors of the invention and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis, et al., BASIC METHODS IN MOLECULAR BIOLOGY (1986) and Sambrook, et al., MOLECULAR CLONING: A LABORATORY MANUAL, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, 293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook, et al., MOLECULAR CLONING, A LABORATORY MANUAL (supra).

For secretion of the translated protein into the lumen of the endoplasmic reticulum, into the periplasmic space or into the extracellular environment, appropriate secretion signals may be incorporated into the desired polypeptide. These signals may be endogenous to the polypeptide or they may be heterologous signals.

If the CXCR4 or SDF-1α polypeptide is to be expressed for use in screening assays, generally, it is preferred that the polypeptide be produced at the surface of the cell. In this event, the cells may be harvested prior to use in the screening assay. If CXCR4 or SDF-1α polypeptide is secreted into the medium, the medium can be recovered in order to recover and purify the polypeptide; if produced intracellularly, the cells must first be lysed before the polypeptide is recovered.

CXCR4 or SDF-1α polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

Screening Assays

The human CXCR4 receptor is found in a variety of tissues and organs in the mammalian host, including vascular endothelium, peripheral blood cells, thymus, spleen, skeletal muscle, heart, brain, liver, colon, lung, placenta, kidney, pancreas, etc., and are responsible for many biological functions, including many pathologies.

Northern blot analysis reveals that the human CXCR4 receptor is selectively expressed in vascular EC and not in smooth muscle cells. (See Example 3.) Moreover, the data discussed in Example 4 indicates that the human CXCR4 receptor is expressed on the surface of vascular EC. Example 5 shows that SDF-1α. is an efficacious attractant of EC, such that SDF-1-α migration of EC. Of the chemokines tested, only SDF-1-α induced a rapid mobilization of intracellular Ca²⁺ in EC. Salcedo, et al., Amer. J. Path. 154(4): 1125-1135 (1999), generated data indicating that rat SDF-1α induces angiogenic sprouting at subnanomolar concentrations from rat aortic rings in the absence of inflammatory cell infiltrates. Therefore, due to EC expression of CXCR4, its ligand, SDF-1α plays a direct role in angiogenesis, thereby modulating the vascular endothelium responses to inflammation, injury and infections, such Human Immunodeficiency Virus (HIV). Accordingly, it is desirous to find compounds and drugs that stimulate or inhibit the function of human CXCR4.

Thus, a preferred embodiment of the present invention relates to a method for inducing chemotaxis in endothelial cells comprising contacting the cells with a chemotaxis inducing effective amount of stromal cell derived factor-1α (SDF 1-α) (SEQ ID NO:4) in combination with a carrier.

Another preferred embodiment of the present invention relates to a method for stimulating angiogenesis in the vasculature of a patient in need thereof comprising contacting the vasculature with an angiogenesis-stimulating-effective amount of SDF 1-α (SEQ ID NO:4) in combination with a carrier.

Yet another preferred embodiment of the present invention relates to a method for stimulating angiogenesis in the vasculature of a patient comprising contacting the vasculature with an angiogenesis-stimulating-effective amount of an agonist of the interaction between CXCR4 (SEQ ID NO:2) and SDF 1-α (SEQ ID NO:4), wherein the patient is suffering from atherosclerosis, restenosis, and spinal cord injury.

Still another preferred embodiment of the present invention relates to a method for inhibiting angiogenesis in the vasculature of a patient comprising contacting the vasculature with an angiogenesis-inhibiting-effective amount of an antagonist of the interaction between CXCR4 (SEQ ID NO:2) and SDF 1-α (SEQ ID NO:4), wherein the patient is suffering from an inflammatory disease, such as rheumatoid arthritis, diabetic retinopathy, inflammatory bowel disease, atherosclerosis, restenosis, stroke, Alzheimer's disease, congestive heart failure, and cardiac remodeling or an angiogenic disease, such as solid tumors (i.e., colon cancer, esophageal cancer, breast cancer, etc.), Kaposi Sarcoma, rheumatoid arthritis, and diabetic retinopathy.

A human CXCR4 receptor polypeptide may be employed in a process for screening for compounds that bind the receptor and that activate (called agonists) or inhibit the activation of (called antagonists) the human CXCR4 polypeptide receptor.

Thus, human CXCR4 polypeptides may also be used to assess the binding of small molecule substrates and ligands in, for example, cells, cell-free preparations, chemical libraries, and natural product mixtures. These substrates and ligands may be natural substrates and ligands or may be structural or functional mimetics. See Coligan, et al., Current Protocols in Immunology 1(2):Chapter 5 (1991).

Human CXCR4 proteins are responsible for many biological functions, including many pathologies. Provided by the invention are screening methods to identify compounds that stimulate or that inhibit the function the function or level of the polypeptide. In general, agonists are employed for therapeutic and prophylactic purposes for such conditions as atherosclerosis, restenosis, ischaemic stroke, and spinal cord injury, whereas antagonists are employed for therapeutic and prophylactic purposes for such conditions as: viral (i.e., Acquired Immunodeficiency Syndrome (AIDS)), bacterial, fungal and protozoan infections, pain, cancer, diabetes, obesity, anorexia, bulimia, asthma, allergies, Parkinson's disease, acute heart failure, hypotension, hypertension, urinary retention, osteoporosis, angina pectoris, myocardial infarction, stroke, ulcers, benign prostatic hypertrophy, migraine, vomiting, psychotic and neurological disorders (i.e., anxiety, schizophrenia, manic depression, depression, delirium, dementia, mental retardation, etc.) and dyskinesias (i.e., Huntington's disease and Gilles de la Tourette's syndrome, etc.), inflammatory diseases, such as rheumatoid arthritis, diabetic retinopathy, inflammatory bowel disease, atherosclerosis, restenosis, stroke, Alzheimer's disease, congestive heart failure, and cardiac remodeling; angiogenic diseases, such as solid tumors (i.e., colon cancer, esophageal cancer, breast cancer, etc.), Kaposi Sarcoma, rheumatoid arthritis, diabetic retinopathy; and spinal cord injury, among others.

In general, such screening procedures involve providing appropriate cells that express the human CXCR4 polypeptide receptor on the surface thereof. Such cells include cells from mammals, yeast, Drosophila or E. coli. In particular, a polynucleotide encoding the receptor of the present invention is employed to transfect cells to thereby express the human CXCR4 polypeptide receptor. The expressed receptor is then contacted with a test compound to observe binding, stimulation or inhibition of a functional response.

One such screening procedure involves the use of melanophores that are transfected to express the human CXCR4 polypeptide receptor. Such a screening technique is described in PCT WO 92/01810, published Feb. 6, 1992. Such an assay may be employed to screen for a compound that inhibits activation of the human CXCR4 receptor polypeptide by contacting the melanophore cells encoding the receptor with both the receptor ligand, SDF-1α polypeptide (SEQ ID NO:4), and a compound to be screened. Inhibition of the signal generated by the ligand indicates that a compound is a potential antagonist for the receptor, i.e., inhibits activation of the receptor.

This technique may also be employed for screening of compounds that activate the receptor by contacting such cells with compounds to be screened and determining whether such a compound generates a signal, i.e., activates the receptor.

Other screening techniques include the use of cells that express the human CXCR4 receptor polypeptide (for example, transfected CHO cells) in a system that measures extracellular pH changes caused by receptor activation. In this technique, compounds may be contacted with cells expressing the receptor polypeptide. A second messenger response, e.g., signal transduction or pH changes, is then measured to determine whether the potential compound activates or inhibits the receptor.

Another screening technique involves expressing the CXCR4 polypeptide in that the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds that are antagonists, and thus inhibit activation of the human CXCR4 polypeptide receptor by determining inhibition of binding of labeled SDF-1α ligand, to cells having the receptor on the surface thereof, or cell membranes containing the receptor. Such a method involves transfecting a eukaryotic cell with DNA encoding the human CXCR4 polypeptide receptor such that the cell expresses the receptor on its surface. The cell is then contacted with a potential antagonist in the presence of a labeled form the SDF-1α ligand. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand that binds to the receptors. This method is called binding assay. Naturally, this same technique can be used to identify an agonist.

Another screening procedure involves the use of mammalian cells (CHO, HEK 293, Xenopus Oocytes, RBL-2H3, etc.) that are transfected to express the receptor of interest. The cells are loaded with an indicator dye that produces a fluorescent signal when bound to calcium, and the cells are contacted with a test substance and a receptor agonist, such as SDF-1α. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening procedure involves use of mammalian cells (CHO, HEK293, Xenopus Oocytes, RBL-2H3, etc.) that are transfected to express the receptor of interest, and that are also transfected with a reporter gene construct that is coupled to activation of the receptor (for example, luciferase or beta-galactosidase behind an appropriate promoter). The cells are contacted with a test substance and the receptor agonist (ligand), such as SDF-1α, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for antagonists or agonists involves introducing RNA encoding the human CXCR4 polypeptide receptor into Xenopus oocytes (or CHO, HEK 293, RBL-2H3, etc.) to transiently or stably express the receptor. The receptor oocytes are then contacted with the receptor ligand, SDF-1α, and a compound to be screened. Inhibition or activation of the receptor is then determined by detection of a signal, such as, cAMP, calcium, proton, or other ions.

Another method involves screening for human CXCR4 polypeptide receptor inhibitors by determining inhibition or stimulation of human CXCR4 polypeptide receptor-mediated cAMP and/or adenylate cyclase accumulation or dimunition. Such a method involves transiently or stably transfecting a eukaryotic cell with human CXCR4 polypeptide receptor to express the receptor on the cell surface. The cell is then exposed to potential antagonists in the presence of human CXCR4 polypeptide receptor ligand, such as SDF-1α. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist binds the receptor, and thus inhibits human CXCR4 receptor polypeptide-ligand binding, the levels of human CXCR4 polypeptide receptor-mediated cAMP, or adenylate cyclase activity, will be reduced or increased.

Another screening method for agonists and antagonists relies on the endogenous pheromone response pathway in the yeast, Saccharomyces cerevisiae. Heterothallic strains of yeast can exist in two mitotically stable haploid mating types, MATa and MATa. Each cell type secretes a small peptide hormone that binds to a G-protein coupled receptor on opposite mating-type cells that triggers a MAP kinase cascade leading to GI arrest as a prelude to cell fusion. Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U.S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclin-dependent kinases leading to cell cycle arrest; and (iii) construction of reporter genes fused to the FUS1 gene promoter (where FUS1 encodes a membrane-anchored glycoprotein required for cell fusion). Downstream reporter genes can permit either a positive growth selection (e.g., histidine prototrophy using the FUS1-HIS3 reporter), or a calorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, et al., Nature 384: 14-16 (1996); Manfredi, et al., Mol. Cell. Biol. 16: 4700-4709 (1996)). This provides a rapid direct growth selection (e.g., using the FUS1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands. Functional agonists of sufficient potency (whether natural or surrogate) can be used as screening tools in yeast cell-based assays for identifying G-protein coupled receptor antagonists. For example, agonists will promote growth of a cell with FUS-HIS3 reporter or give positive readout for a cell with FUS 1-LacZ. However, a candidate compound that inhibits growth or negates the positive readout induced by an agonist is an antagonist. For this purpose, the yeast system offers advantages over mammalian expression systems due to its ease of utility and null receptor background (lack of endogenous G-protein coupled receptors), which often interferes with the ability to identify agonists or antagonists.

Another embodiment of the present invention relates to the agonists and antagonists obtainable from the above described screening methods. Examples of potential human CXCR4 polypeptide receptor antagonists include peptidomimetics, synthetic organic molecules, natural products, antibodies, etc., that bind to the receptor, but do not elicit a second messenger response, such that the activity of the receptor is prevented.

Potential antagonists also include proteins that are closely related to the ligand of the human CXCR4 polypeptide receptor, i.e., a fragment of the ligand, which have lost biological function, and when they bind to the human CXCR4 polypeptide receptor, elicit no response.

Thus in another aspect, the present invention relates to a screening kit for identifying agonists, antagonists, and ligands for the human CXCR4 polypeptide receptor, that comprises:

(a) a human CXCR4 polypeptide receptor, preferably that of SEQ ID NO: 1; and further preferably comprises labeled or unlabeled SDF-1α preferably that of SEQ ID NO:2;

(b) a recombinant cell expressing a human CXCR4 polypeptide receptor, preferably that of SEQ ID NO:1; and further preferably comprises labeled or unlabeled SDF-1α, preferably that of SEQ ID NO:2; or

(c) a cell membrane expressing human CXCR4 polypeptide receptor; preferably that of SEQ ID NO:1; and further preferably comprises labeled or unlabeled SDF-1α, preferably that of SEQ ID NO:2.

It will be appreciated that in any such kit, (a), (b), or (c) may comprise a substantial component.

As noted above, a potential antagonist is a small molecule that binds to the human CXCR4 polypeptide receptor, making it inaccessible to its ligand, SDF-1α, such that normal biological activity is prevented. Examples of small molecules include, but are not limited to, small peptides or peptide-like molecules.

Potential antagonists also include soluble forms of the human CXCR4 polypeptide receptor, e.g., fragments of the receptor, that bind to the ligand and prevent the ligand from interacting with membrane bound human CXCR4 polypeptide receptors.

Potential antagonists also include soluble forms of a human CXCR4 polypeptide, e.g., fragments of the polypeptide, that bind to the ligand and prevent the ligand from interacting with membrane bound human CXCR4 polypeptides. Potential antagonists also include antibodies that bind to the SDF-1α ligand and prevent the ligand from binding or activating the human CXCR4 receptor.

In a preferred embodiment of the present invention, the antagonist compounds of the interaction between CXCR4 and SDF1-α include, but are not limited to:

1-[4-(1,5-Diazacyclooctan-1-ylmethyl)phenylmethyl]-1,4,8,11-tetraazacyclotetradecane hexahydrochloride;

1-[4-(2-Guanidinobenzimidazol-1-ylmethyl)phenylmethyl]-1,4,8,11-tetraazacyclotetradecane pentahydrochloride;

1-[4-(5,6,14,15-Dibenzo-1,4-dioxa-8,12-diazacyclopentadeca-5,14-dien-8-ylmethyl)phenylmethyl]-1,4,8,11-tetraazacyclotetradecane hexahydrochloride;

1-[4-(Azacyclotridecan-1-ylmethyl)phenylmethyl]-1,4,8,11-tetraazacyclotetradecane pentahydrochloride; and

1-[4-(1,4-Diazacycloheptan-1-ylmethyl)phenylmethyl]-1,4,8,11-tetraazacyclotetradecane hexahydrochloride.

The above-referenced compounds are prepared by methods analogous to that shown in Scheme 1.

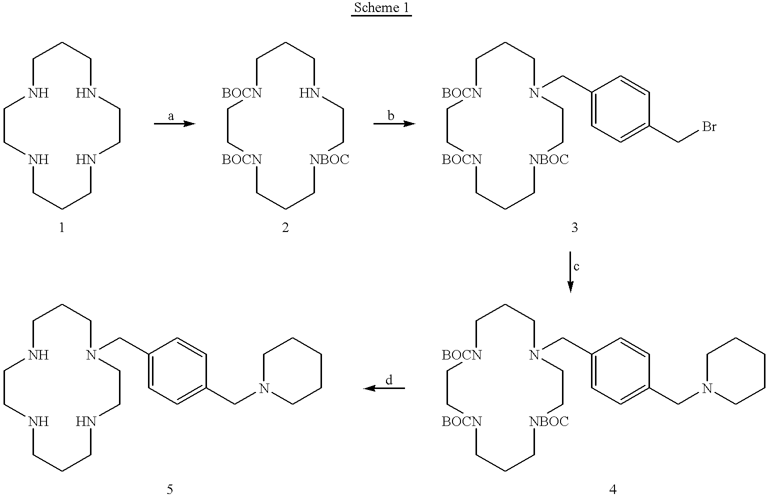

a) BOC₂O, CH₂Cl₂; b) α,α′-dibromo-p-xylene, K₂CO₃, MeCN, 60° C.; c) piperidine, K₂CO₃, MeCN, 60° C.; d) HCl, dioxane, CH₂Cl₂.

Compound 1, available commercially, is protected as its tri-tert-butylcarbamate derivative 2, which is alkylated on the free nitrogen to give compound 3. The benzylic bromide is displaced with the appropriate N nucleophile to give the protected precursor 4, which is deprotected with acid to furnish the final compound 5.

In another preferred embodiment of the present invention, the antagonist compounds useful in the present invention include, but are not limited to:

1-(4-{Bis[2-(diethylamino)ethyl]aminomethyl}phenylmethyl)-1,4,8,11-tetraazacyclotetradecane heptahydrochloride;

1-(4-{[(2-Aminoethyl)(3-aminopropyl)amino]methyl]}phenylmethyl)-1,4,8,11-tetraazacyclotetradecane heptahydrochloride;

1-{4-[Di-(2-pyridyl)aminomethyl]phenylmethyl}-1,4,8,11-tetraazacyclotetradecane pentahydrochloride; and

[4-(2-Thiazolylaminomethyl)phenylmethyl]-1,4,8,11-tetraazacyclotetradecane pentahydrochloride.

These compounds are prepared by methods analogous to that shown in Scheme 2.

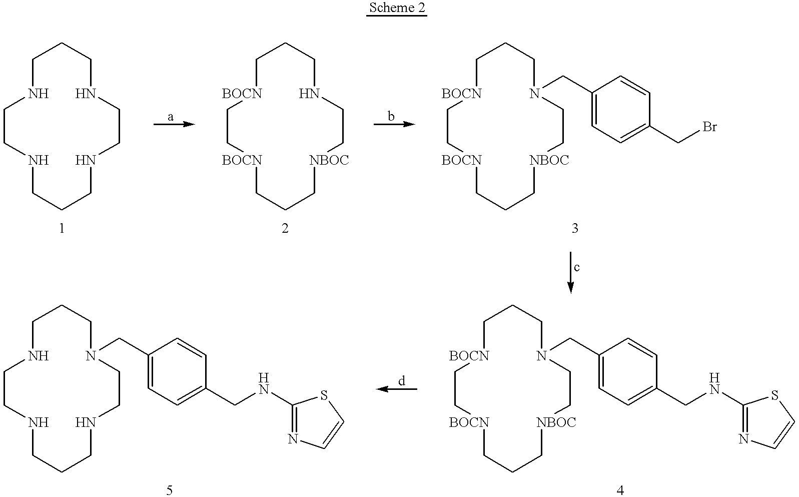

a) BOC₂O, CH₂Cl₂; b) α,α′-dibromo-p-xylene, K₂CO₃, MeCN, 60° C.; c) 2-aminothiazole, K₂CO₃, MeCN, 60 C; d) HCl, dioxane, CH₂Cl₂.

Compound 1, available commercially, is protected as its tri-tert-butylcarbamate derivative 2, which is alkylated on the free nitrogen to give compound 3. The benzylic bromide is displaced with the appropriate N nucleophile to give the protected precursor 4, which is deprotected with acid to furnish the final compound 5.

In yet another preferred embodiment of the present invention, the antagonist compounds useful in the present invention include, but are not limited to:

1,4-Bis[2-(2-benzimidazolylamino)-5,5-di(2-pyridyl)-4-oxo-5H-imidazolin-3-ylmethyl]benzene bis-trifluoroacetic acid salt;

2,6-Bis[2-(2-benzimidazolylamino)-5,5-di(2-pyridyl)-4-oxo-5H-imidazolin-3-ylmethyl]pyidine bis-trifluoroacetic acid salt; and

1,4-Bis{[1-(2-Benzimidazolyl)-1-guanidino]methyl}benzene.

These compounds are prepared by methods analogous to that shown in Scheme 3.

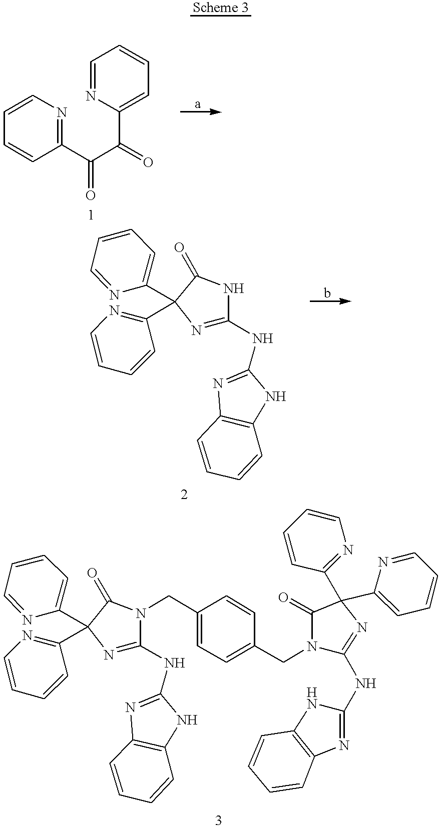

a) 2-Guanidinobenzimidazole, NaOH, rt; b) α,α′-dibromo-p-xylene, DMF, rt Compound 1, available commercially, condenses with 2-guanidinobenzimidazole to give the rearranged product 2, which is converted to its sodium salt and alkylated regioselectively with a bis-electrophile to give compound 3.

Also included in these antagonist compounds are pharmaceutically acceptable salts and complexes of all of the above-refenenced compounds. Preferred are the zinc, copper, nickel, cobalt and rhodium complexes, hydrochloride, hydrobromide and trifluoroacetate salts. These antagonists may contain one or more asymmetric carbon atoms and may exist in racemic and optically active forms. All of these compounds and diastereomers are contemplated to be within the scope of the antagonists of the present invention.

The above-referenced antagonists were identified by the assay disclosed in Example 8. The methods of preparation of each of the above-referenced antagonists are exemplified in Examples 9-21.

Prophylactic and Therapeutic Methods

This invention provides methods of treating an abnormal conditions related to both an excess of and insufficient amounts of human CXCR4 receptor or SDF-1α ligand activity.

If the activity of human CXCR4 receptor is in excess, several approaches are available. One approach comprises administering to a subject an inhibitor compound (antagonist) as hereinabove described along with a pharmaceutically acceptable carrier in an amount effective to inhibit activation by blocking binding of ligands to the human CXCR4 receptor, or by inhibiting a second signal, and thereby alleviating the abnormal condition.

In another approach, soluble forms of human CXCR4 polypeptides still capable of binding the ligand in competition with endogenous human CXCR4 may be administered. Typical embodiments of such competitors comprise fragments of the human CXCR4 polypeptide.

In still another approach, expression of the gene encoding endogenous human CXCR4 can be inhibited using expression blocking techniques. Known such techniques involve the use of antisense sequences, either internally generated or separately administered. See, for example, O'Connor, J. Neurochem. 56:560 (1991) in Oligodeoxynucleotides as Antisense Inhibitors of Gene Expression, CRC Press, Boca Raton, Fla. (1988). Alternatively, oligonucleotides that form triple helices with the gene can be supplied. See, for example, Lee, et al., Nucleic Acids Res 6:3073 (1979); Cooney, et al., Science 241:456 (1988); Dervan, et al., Science 251:1360 (1991). These oligomers can be administered per se or the relevant oligomers can be expressed in vivo.

For treating abnormal conditions related to an under-expression of human CXCR4 receptor and its activity, several approaches are also available. One approach comprises administering to a subject a therapeutically effective amount of a compound that activates human CXCR4 receptor, i.e., an agonist as described above, in combination with a pharmaceutically acceptable carrier, to thereby alleviate the abnormal condition. Alternatively, gene therapy may be employed to effect the endogenous production of human CXCR4 receptor by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd. (1996).

Formulation and Administration

Peptides, such as the soluble form of human CXCR4 or SDF-1α polypeptides, and agonists and antagonist peptides or small molecules, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, and combinations thereof. Formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like.

The dosage range required depends on the choice of peptide, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Biological Methods

### EXAMPLES

### Example 1

### Materials Cells, and Culture Conditions

Recombinant human IFN-γ, TNF-α, IL-1β and TGF-β were purchased from Genzyme (Cambridge, Mass.). Bacterial LPS, actinomycin D and DMSO were from Sigma Chemical Co. (St. Louis, Mo.). SDF-1α was obtained from Gryphon Sciences, San Francisco, Calif., and other chemokines were from R&D Systems (Minneapolis, Minn.).

Primary cultures of HUVEC and human coronary artery endothelial cells (HCAEC) were purchased from Cell Systerms (Kirkland, Wash.) and maintained in their proprietary CS-C complete medium without antibiotics, in tissue culture flasks coated with 0.1% gelatin (Sigma Chemical Co., St. Louis, Mo.). Cells were passaged at confluence and used within the first seven passages.

### Example 2

### Human CXCR4 Receptor Expression in EC

Based on the published sequence of human chemokine receptors, the following pair of consensus degenerate 20-mer primers were synthesized from the ends of the 3rd and 7th transmembrane domains of chemokine receptors.

CK-F: 5′-TAY-CTS-GCY-ATY-GTS-CAY-GC-3′ (SEQ ID NO:5)

CK-R: 5′-AAR-GCR-TAR-ATS-AYK-GGR-TT-3′ (SEQ ID NO:6)

The symbols follow the IUB/GCG convention (Y=C/T, S=C/G, R=A/G, K=G/T).

Total cellular RNA was isolated from 10⁷ early passage HUVEC and HCAEC by the single extraction Tri-reagent procedure (Molecular Research Center, Inc. Cincinnati, Ohio), according to the manufacturers protocol and stored dissolved in Formazol at −80° C. PCR amplification of total RNA was done with the GeneAmp RNA PCR kit (Perkin Elmer, Norwalk, Conn.), as described by Gupta, et al., Gene 124: 287-290.35 (1993). Two μg of total RNA was reverse transcribed with the “downstream” antisense oligomer, CK-R (SEQ ID NO:6). The “upstream” oligomer CK-F (SEQ ID NO:5), was added directly to the reaction tubes along with the PCR “reaction mix” and subjected to 35 cycles of amplification. Each cycle consisted of 1 minute denaturation at 95° C., annealing at 55° C. for 1 minute and elongation at 60° C. for 2 minutes. The final extension step lasted 7 min. at 72° C. The PCR products were analyzed on agarose gels and subcloned directly into the PCR^{II} TA vector (Invitrogen, Carlsbad, Calif.). Plasmid DNA from individual colonies were analyzed by restriction digestion and sequencing.

To explore the expression of chemokine receptor transcripts in human EC, total cellular RNA from HUVEC and HCAEC was amplified by RT-PCR. The expected 515-base pair product was amplified, and the product from HUVEC was subcloned to generate a cDNA plasmid library enriched for chemokine receptor clones. The restriction analysis of representative clones, which were later identified as containing inserts having CXCR4, CXCR2, CCR3 and unknown sequences. 110 out of the 250 isolated clones were randomly sequenced. CXCR4, representing 45% of the sequenced clones was the most prevalent chemokine receptor, followed by clones with identity to CCR3 (10%), the eotaxin receptor. Also present were clones having inserts with CXCR1, CCR1 and CCR2 sequences. These data provide evidence that vascular EC have the ability to express mRNA for several chemokine receptors. The results are also consistent with previous reports where CXCR2 expression was detected in HUVEC by RT-PCR (Schonbeck, et al., J. Immunol. 154: 2375-2383 (1995)), and specific binding of IL-8 and RANTES was observed on the endothelium of postcapillary venules and veins in human skin by using an in situ binding assay. Rot, et al., J. Leuk. Biol. 59:39-44 (1996).

### Example 3

### Northern Blot Analysis

Total RNA (10 μg/lane) was fractionated on 1% agarose-formaldehyde gels, transferred to a nylon membrane (Amersham Corp., Piscataway, N.J.) and covalently linked with a UV crosslinker (Stratagene Inc., La Jolla, Calif.). For Northern analysis, 515-base pair size cDNA probes of CXCR1, CXCR2, CXCR3, CXCR4, CCR1, CCR2 and CCR3, were used. The GAPDH gene probe (Clontech, Palo Alto, Calif.) was used to normalize RNA sample differences in each lane. The probes were labeled with [a-³²P] dCTP using a random-prime labeling kit (Promega Corp., Madison, Wis.), and hybridized overnight at 42° C. in 6× SSC buffer (1× SSC=150 mM NaCL, 15 mM Na Citrate), 0.1% sodium dodecyl sulfate, 5× Denhardt's solution, 50% formamide, and 100 μg/ml denatured salmon sperm DNA. Membranes were washed with a final stringency of 0.2× SSC at 60° C., and analyzed with a phosphorimager (Molecular Dynamics, Inc., Sunnyvale, Calif.) after exposure at room temperature for 3-5 days. Densitometry was used for quantitative analysis.

Steady state expression of chemokine receptors in vascular EC was studied by Northern blot analysis of total RNA. Both HUVEC and HCAEC expressed similar amounts of an expected 1.8 Kb size mRNA after hybridization with the CXCR4 cDNA probe. These results also indicate that CXCR4 is the most abundant chemokine receptor expressed in vascular EC, as identical Northern blots with EC RNA did not hybridize with 515-base pair size CXCR1, CXCR2, CXCR3, CCR1, CCR2 and CCR3 cDNA probes.

CXCR4 transcripts are well expressed in many non-hematopoietic vascular tissues like heart, brain, lung and colon. Federsppiel, et al. Genomics 16: 707-712 (1993). However, at the cellular level, this expression was selective for EC, as indicated by the failure of total RNA from human pulmonary artery smooth muscle cells (HPASMC) to hybridize with the CXCR4 cDNA probe. To understand the regulation of CXCR4 in EC during inflammation, we treated the HUVEC with various mediators and measured its steady state mRNA levels after normalization against the GAPDH cDNA probe. IFN-γ and, to a lesser extent, TNF-α, caused a decrease in CXCR4 mRNA levels after 24 hours of treatment. IL-1 and LPS caused a significant induction, while no effect was observed after treatment with TGF-β, γIP-10 and DMSO. The transcription inhibitor, actinomycin D caused an almost complete abrogation of CXCR4 message in the same time period.

### Example 4

### Human CXCR4 Receptor is Expressed on EC Surface

Cell surface expression of CXCR4 receptors was analyzed as described by Bleul, et al. Nature 382: 829-833 (1996) and Gupta, et al. Gene 124: 287-290 (1993). Briefly, 5×10⁵ HUVEC were permeabilized in the presence of 0.2% Triton X-100/PBS for 2 minutes, and then resuspended in an ice-cold PBS, 0.1% bovine serum alubumin. Cells were incubated on ice for 30 minutes with the primary 12G5 antibody (Gupta, et al., Gene 124: 287-290.35 (1993)) or a control antibody (R&D Systems, Minneapolis, Minn.) of the same subclass. Cells were then washed twice with ice-cold PBS, 0.1% bovine serum albumin and labeled with a second-stage fluorescin isothiocyanate-conjugated goat anti-mouse IgG (Biosource International, Camarillo, Calif.). FACS analysis was done with a FACScan flow cytometer (Becton Dickinson, Franklin Lakes, N.J.).

The cell surface expression of human CXCR4 receptor was evaluated by FACS analysis of HUVEC by using the specific monoclonal antibody 12G5 as previously described by Endres, et al., Cell 87: 745-756 (1996)). A shift was observed in the fluorescence intensity of cells after treatment with 12G5, indicating that mRNA expression of CXCR4 is translated into surface expression of the receptor on HUVEC.

### Example 5

### SDF-1α Elicits a Ca⁺² Response From EC and is an Efficacious and Potent Chemoattractant

HUVEC migration assay was performed using 5×10⁵ cells/well (in CS-C medium) in the top chamber of a 6.5 mm diameter, 8-μM pore polycarbonate Transwell culture insert (Costar, Cambridge, Mass.) as reported previously (36). Incubation was carried out at 37° C. in 5% CO₂ for 20 hrs. After incubation, migrated cells in the lower chamber were counted with a ZM Coulter counter (Coulter Diagnostics, Hialeah, Fla.). Percent migration was calculated based on the total initial input cells per well.

For measurements of intracellular calcium [Ca²⁺]ᵢ, EC were loaded with 2 μM fura-2/AM (Molecular Probes, Eugene, OR), rinsed with 1 mM EDTA in Dulbecco's PBS, and resuspended into Kreb's Ringer's Henseleit (KRH) buffer, pH 7.4, containing 0.1% gelatin. Cells (1×10⁶/ml) were stored on ice and diluted for use 1:1 with fresh KRH buffer at 37° C. Fluorescence of fura-2 in cells was measured with a University of Pennsylvania Biomedical Instruments Group dual channel fluorometer. Data was captured as voltage recordings with the aid of a PC and analyzed by Igor version 1.28 software (WaveMetrics, Lake Oswego, Oreg.). Chemokines were added from concentrated stocks in water. To establish the integrity of EC, we also measured [Ca²⁺]ᵢ stimulated by thrombin.

To determine whether the human EC express a functional CXCR4 receptor, our subsequent studies used SDF-1α along with several other chemokines to assess their ability to induce changes in intracellular levels of Ca⁺² and cause migration. SDF-1α elicited a rapid, though variable elevation of [Ca⁺²]ᵢ in HUVEC, with maximal response at a concentration of 100 nM. In contrast, other chemokines like γ-IP10, IL-8, PF-4, MIP-1α, MCP-1, eotaxin and RANTES had no effect on EC. These data suggest that EC possess receptors for SDF-1α that are functionally coupled to Ca⁺².

The Applicant next studied the chemotactic response of EC to SDF-1α. SDF-1α induced a pronounced migration of −40% of input EC in a concentration-related manner with an EC₅₀ of 10-20 nM. It is intriguing to observe the high percentage of EC that migrated in response to SDF-1α, even though EC have limited migratory capability in comparison with neutrophils and monocytes. In contrast with other EC chemo-attractants like vitronectin, the chemotactic response to SDF-1α was kinetically robust, and a majority of the migrated cells entered the lower chamber without adhering to the Transwell filter. In these experiments, other chemokines like γ-IP10, IL-8, MIP-1α, MCP-1, eotaxin and RANTES had no effect on EC chemotaxis. Taken together, these observations have obvious biological significance, in that they indicate that SDF-1α plays a role in re-endothelialization after injury, an event that requires the directed migration of EC.

### Example 6

### Determination of Half-Life of CXCR4 Transcripts

In order to understand the kinetics of inflammation mediated transcriptional regulation of CXCR4, actinomycin D was used to determine the half-life of CXCR4 mRNA. Selective degradation of existing mRNA upon addition of actionmycin D to EC cultures indicates that CXCR4 mRNA has a short half-life of around 2 hours and is, therefore, subject to a rapid turnover. In addition, the Applicant observed that actinomycin D had the unexpected effect of sharply increasing the steady state levels of CXCR4 mRNA after a short term exposure of only 15-30 minutes. Many cytokines and cytokine receptors, including CXCR4, have A-U rich elements in their untranslated regions that serve as targeting motifs for transcript degradation by specific RNAses. Shaw, et al., Cell 46: 659-667 (1986). In addition to its action as a transcriptional inhibitor, actinomycin D also has the unique and immediate effect of imparting stability to existing transcripts of mRNA undergoing rapid turnover.

### Example 7

### Upregulation of CXCR4 mRNA in Stoke Model

CXCR4 mRNA was upregulated 5-20 fold in an ischaemic injury induced rat stroke model in a time-dependent manner from within 1 hour after ischaemic injury and up to 15 days after injury. The middle cerebral artery occlusion (MCAO) ischaemia induced injury model in the rat is a well studied model in the art (Barone, et al. Stroke 28: 1233-1244 (1997)).

### Example 8

### CXCR-4/SDF-1α Assay Protocol

Assay plates were seeded with RBL transfected with SDF-1α (SEQ ID NO:4). Dye loading buffer (EMEM w/Earl's salts w/L-glutamine with 1× Sulphinpyrozone and 10% BSA, 100 •L) was added to each well, and the plate incubated for 90 minutes at 37° C. The dye loading buffer was aspirated from the plates. Hydrolysis buffer (EMEM w/Earl's salts w/L-glutamine with 1× Sulphinpyrozone, 100 •L) was added to each well, and the plate incubated for 10 minutes at 37° C. The cells were washed 3 times with wash buffer (1× Krebs Ringer, 15 mM HEPES, 1 mM MgCl, 1 mM CaCl with 1× Sulphinpyrozone and 0.10% gelatin), then wash buffer was dispensed to each well (100 uL/well). The plate was incubated for 10 minutes at 37° C., then placed in FLIPR™ (Molecular Devices). Test compounds in gelatin buffer (1× Krebs Ringer, 15 mM HEPES, 1 mM MgCl, 1 mM CaCl with 0.10% gelatin, 50 uL) were preincubated with cells for 3 minutes, then ligand (SDF-1alpha/PBSF, 15 nM final concentration) was added. The plate was incubated for 2 minutes while continually reading.

Synthetic Chemistry

### Example 9

### Preparation of 1-[4-(4-acetyl-1-piperazinomethyl)phenylmethyl]-1,4,8,11-tetraazacyclotetradecane Pentahydrochloride

a) 1-[4-(bromomethyl)phenylmethyl]-4,8,11-tri-(t-butoxycarbonyl)-1,4,8,11-tetraazacyclotetradecane

a,a′-Dibromo-p-xylene (36.0 g, 136 mmol) was stirred at 60° C. in acetonitrile (500 mL) until it dissolved. Potassium carbonate (3.5 g, 25.3 mmol) was added, followed by the dropwise addition of a solution of 1,4,8-tri-(t-butyloxycarbonyl)-1,4,8,11-tetraazacyclotetradecane (Boitrel, et. al., Tetrahedron Lett., 1995, 36, 4995) (6.0 g, 11.98 mmol) in acetonitrile (100 mL). The mixture was stirred for 6 hours, cooled and partially evaporated. The excess dibromoxylene was filtered off, the mother liquors evaporated under vacuum and chromatographed (silica gel, 50% dichloromethane/hexane to 2% methanol/dichloromethane) to afford the title compound as a foam (7.4 g, 90%). MS (ES+) m/e 683 and 685 [M+H]⁺.

b) 1-[4-(4-acetyl-1-piperazinomethyl)phenylmethyl]-4,8,11-tri-(t-butoxycarbonyl)-1,4,8,11-tetraazacyclotetradecane

A mixture of 1-[4-(bromomethyl)phenylmethyl]-4,8,11-tri-(t-butoxycarbonyl)-1,4,8,11-tetraazacyclotetradecane (326 mg, 0.477 mmol), 1-acetylpiperazine (95 mg, 0.741 mmol) and anhydrous potassium carbonate (350 mg, 2.53 mmol) in acetonitrile (30 mL) was vigorously stirred together at 50° C. for 1 hour. The solvent was evaporated and the residue was purified by flash chromatography (silica gel, 0-3% methanol/dichloromethane) to give the title compound as an oil (300 mg, 86%). MS (ES+) m/e 731 [M+H]⁺.

c) 1-[4-(4-acetyl-1-piperazinomethyl)phenylmethyl]-1,4,8,11-tetraazacyclotetradecane Pentahydrochloride

To a solution of 1-[4-(4-acetyl-1-piperazinomethyl)phenylmethyl]-4,8,11-tri-(t-butoxycarbonyl)-1,4,8,11-tetraazacyclotetradecane (175 mg, 0.239 mmol) in 1,4-dioxane (1.0 mL) was added a solution of 4M hydrogen chloride in 1,4-dioxane (1.0 mL). The mixture was stood for 2 hours, the white solid collected and washed successively with 1,4-dioxane, diethyl ether and hexane.

The hygroscopic solid was dried in vacuo (80° C.) to give the title compound (35 mg, 34%).

MS (ES+) m/e 431 [M+H]⁺

### Example 10

### Preparation of 1-[4-(1,4-diazacycloheptan-1-ylmethyl)phenylmethyl]-1,4,8,11-tetraazacyclotetradecane Hexahydrochloride

Following the procedure of Example 9(a)-(c), except substituting homopiperazine for 1-αcetylpiperazine, the title compound was prepared (27% overall). ¹H NMR (300 MHz, d3-MeOD/D₂O) δ 7.67 (d, 2H), 7.50 (d, 2H), 4.52 (s, 2H), 3.98 (s, 2H), 3.90 (s, 2H), 3.62 (m, 2H), 3.53-3.20 (m, 8H), 3.15 (m, 2H), 2.95 (m, 4H), 2.35 (m, 2H), 2.19 (m, 4H), 2.09 (m, 2H), 1.26 (m, 4H).

### Example 11

### Preparation of 1-[4-(azacycloheptan-1-ylmethyl)phenylmethyl]-1,4,8,11-tetraazacyclotetradecane Pentahydrochloride

Following the procedure of Example 9(a)-(c), except substituting hexamethyleneimine for 1-αcetylpiperazine, the title compound was prepared (45% overall). MS (ES+) m/e 402 [M+H]⁺.

### Example 12

### Preparation of 1-[4-(5,6,14,15-dibenzo-1,4-dioxa-8,12-diazacyclopentadeca-5,14-dien-8-ylmethyl)phenylmethyl]-1,4,8,11-tetraazacyclotetradecane Hexahydrochloride

Following the procedure of Example 9(a)-(c), except substituting 5,6,14,15-dibenzo-1,4-dioxa-8,12-diazacyclopentadeca-5,14-diene for 1-αcetylpiperazine, the title compound was prepared (42% overall). ¹H NMR (300MHz, d6-DMSO, D₂O) δ 7.90-6.95(m, 12H), 4.7-4.1 (m, 14H), 3.5 (s, 2H), 3.45 (s, 2H), 3.5-3.0 (br m, 6H), 2.85-2.30 (br m, 2H), 2.3-2.0 (br m, 6H), 1.2 (br s, 6H,).

### Example 13

### Preparation of 1-[4-(2-guanidinobenzimidazol-1-ylmethyl)phenylmethyl]-1,4,8,11-tetraazacyclotetradecane Pentahydrochloride

a) 1-[4-(2-guanidinobenzimidazol-1-ylmethyl)phenylmethyl]-4,8,11-tri-(t-butoxycarbonyl)-1,4,8,11-tetraazacyclotetradecane

A mixture of 1-[4-(bromomethyl)phenylmethyl]-4,8,11-tri-(t-butoxycarbonyl)-1,4,8,11-tetraazacyclotetradecane (293 mg, 0.429 mmol) and 2-guanidinobenzimidazole (225 mg, 1.28 mmol) in acetonitrile (5 mL) was stirred and heated under reflux for 30 min. The solvent was evaporated and the residue was purified by flash chromatography (silica gel, 0-5% methanol/dichloromethane) to give the title compound as a yellow gum, (115 mg, 34%). MS (ES+) m/e 778 [M+H]⁺.

b) 1-[4-(2-guanidinobenzimidazol-1-ylmethyl)phenylmethyl]-1,4,8,11-tetraazacyclotetradecane Pentahydrochloride

To a solution of 1-[4-(2-guanidinobenzimidazol-1-ylmethyl)phenylmethyl]-4,8,11-tri-(t-butoxycarbonyl)-1,4,8,11-tetraazacyclotetradecane (115 mg, 0.148 mmol) in 1,4-dioxane (2.0 mL) was added a 4M solution of hydrogen chloride in 1,4-dioxane (1.5 mL). The mixture was stood overnight, the red solid collected and washed successively with 1,4,-dioxane, diethyl ether and hexane. The hygroscopic solid was dried in vacuo (80° C.) to give the title compound (72 mg, 73%). MS (ES+) m/e 478 [M+H]⁺.

### Example 14

### Preparation of 1-[4-(1,5-diazacyclooctan-1-ylmethyl)phenylmethyl]-1,4,8,11-tetraazacyclotetradecane Hexahydrochloride

Following the procedure of Example 9(a)-(c), except substituting 1,5-diazacyclooctane dihydrobromide (Ewin, et al., J. Chem. Res., Synop., 1985, 11, 334) for 1-αcetylpiperazine, the title compound was prepared (6% overall). ¹H NMR (300 MHz, d6-DMSO/D₂O) δ 7.74 (s, 4H), 4.43 (br s, 4H), 3.6-3.0 (br m, 20H), 2.21 (br m, 12H).

### Example 15

### Preparation of 1-(4-{bis[2-(diethylamino)ethyl]aminomethyl}phenylmethyl)-1,4,8,11-tetraazacyclotetradecane Heptahydrochloride

a) 1-[4-(bromomethyl)phenylmethyl]-4,8,11-tri-(t-butoxycarbonyl)-1,4,8,11-tetraazacyclotetradecane

a,a′-Dibromo-p-xylene (36.0 g, 136 mmol) was stirred at 60° C. in acetonitrile (500 mL) until it dissolved. Potassium carbonate (3.5 g, 25.3 mmol) was added, followed by the dropwise addition of a solution of 1,4,8-tri-(t-butyloxycarbonyl)-1,4,8,11-tetraazacyclotetradecane (Boitrel et. al., Tetrahedron Lett., 1995, 36, 4995) (6.0 g, 11.98 mmol) in acetonitrile (100 mL). The mixture was stirred for 6 hours, cooled and partially evaporated. The excess dibromoxylene was filtered off, the mother liquors evaporated under vacuum and chromatographed (silica gel, 50% dichloromethane/hexane to 2% methanol/dichloromethane) to afford the title compound as a foam (7.4 g, 90%). MS (ES+) m/e 683 and 685 [M+H]⁺.

b) 1-(4-{bis[2-(diethylamino)ethyl]aminomethyl}phenylmethyl)-4,8,11-tri-(t-butoxycarbonyl)-1,4,8,11-tetraazacyclotetradecane

A mixture of 1-[4-(bromomethyl)phenylmethyl]-4,8,11-tri-(t-butoxycarbonyl)-1,4,8,11 tetraazacyclotetradecane (279 mg, 0.408 mmol), N,N,N′,N′-tetraethyldiethylenetriamine (211 uL, 0.820 mmol) and anhydrous potassium carbonate (100 mg, 0.724 mmol) in acetonitrile (5 mL) was vigorously stirred together at 50° C. for 1 hour. The solvent was evaporated and the residue was purified by flash chromatography (silica gel, 0-5% methanol/dichloromethane) to give the title compound as an oil, (210 mg, 63%) MS (ES+) m/e 818 [M+H]⁺.

c) 1-(4-{bis[2-(diethylamino)ethyl]aminomethyl}phenylmethyl)-1,4,8,11-tetraazacyclotetradecane Heptahydrochloride

To a solution of 1-(4-{bis[2-(diethylamino)ethyl]aminomethyl}phenylmethyl)-4,8,11-tri-(t-butoxycarbonyl)-1,4,8,11-tetraazacyclotetradecane (200 mg, 0.244 mmol) in 1,4-dioxane (2.0 mL) was added a 4M solution of hydrogen chloride in diethyl ether (1.5 mL). The mixture was stood overnight, the solid collected and washed successively with diethyl ether and hexane.

The hygroscopic solid was dried in vacuo (80° C.) to give the title compound (100 mg, 53%). ¹H NMR (300 MHz, d6-DMSO/D₂O) δ 7.6 (m, 4H), 4.0-2.9 (br m, 24H), 2.2 (br s, 4H), 1.11 (m, 12H), 1.07 (t, 12H).

### Example 16

### Preparation of 1-{4-[(2-aminoethyl)(3-aminopropyl)aminomethyl]phenylmethyl}-1,4,8,11-tetraazacyclotetradecane Heptahydrochloride

a) (2-phthalimidoethyl)(3-phthalimidoprop-1-yl)amine

A mixture of N-(2-aminoethyl)-1,3-propanediamine (10.0 mL, 79.2 mmol), phthalic anhydride (24.6 g, 166 mmol) and p-toluenesulfonic acid (1.0 g, 5.26 mmol) in toluene (500 mL) was stirred and heated under reflux, using a Dean & Stark head, for 5 hours. The mixture was cooled and diluted with hexane. The solid was collected, washed with ether and hexane, and dried to give the title compound as a pale yellow solid (21 g, 72%). MS (ES+) m/e 378 [M+H]⁺.

b) 1-[4-{[(2-phthalimidoethyl)(3-phthalimidoprop-1-yl)amino]methyl}phenylmethyl]-1,4,8,11-tetraazacyclotetradecane Pentahydrochloride

Following the procedure of Example 15(a)-(c), except substituting (2-phthalimidoethyl)(3-phthalimidoprop-1-yl) amine for N,N,N′,N′-tetraethyldiethylenetriamine, the title compound was prepared (58% overall). MS (ES+) m/e 341 [M+2H]2+.

### Example 17

### 1-{4-[di-(2-pyridyl)aminomethyl]phenylmethyl}-1,4,8,11-tetraazacyclotetradecane Pentahydrochloride

a) 1-{4-[di-(2-pyridyl)aminomethyl]phenylmethyl}-4,8,11-tri-(t-butoxycarbonyl)-1,4,8,11-tetraazacyclotetradecane

2,2′-Dipyridylamine (77 mg, 0.500 mmol) was added to a suspension of sodium hydride (18.8 mg of a 60% dispersion in mineral oil, 0.470 mmol) in anhydrous DMF (10 mL) and the mixture stirred at 25° C. for 1 hour under nitrogen. A solution of 1-[4-(bromomethyl)phenylmethyl]-4,8,11-tri-(t-butoxycarbonyl)-1,4,8,11-tetraazacyclotetradecane 1194 (292 mg, 0.427 mmol) in anhydrous DMF was added and the mixture stirred overnight. Water was added and the mixture extracted twice with diethyl ether and once with ethyl acetate. The combined organic extracts were washed with water, dried (anhydrous potassium carbonate) and evaporated to a yellow gum, which was purified by flash chromatography (silica gel, 0-5% methanol/dichloromethane) to give the title compound as an oil, (100 mg 30%). MS (ES+) m/e 774 [M+H]⁺.

b) 1-{4-[di-(2-pyridyl)aminomethyl]phenylmethyl}-1,4,8,11-tetraazacyclotetradecane Pentahydrochloride

Following the procedure of Example 15(c), except substituting 1-{4-[di-(2-pyridyl)aminomethyl]phenylmethyl}-4,8,11-tri-(t-butoxycarbonyl)-1,4,8,11-tetraazacyclotetradecane for 1-(4-{bis[2-(diethylamino)ethyl]aminomethyl}phenylmethyl)-4,8,11-tri-(t-butoxycarbonyl)-1,4,8,11-tetraazacyclotetradecane, the title compound was prepared (94%). MS (ES+) m/e 474 [M+H]⁺.

### Example 18

### Preparation of 1-[4-(2-thiazolylaminomethyl)phenylmethyl]-1,4,8,11-tetraazacyclotetradecane Pentahydrochloride

Following the procedure of Example 15(a)-(c), except 2-aminothiazole for N,N,N′,N′-tetraethyldiethylenetriamine, the title compound was prepared (19% overall). MS (ES+) m/e 202 [M+2H]²⁺.

### Example 19

### Preparation of 1,4-bis[2-(2-benzimidazolylamino)-5,5-di(2-pyridyl)-4-oxo-5H-imidazolin-3-ylmethyl]benzene bis-trifluoroacetic Acid Salt

a) 2-(2-benzimidazolylamino)-5,5-di(2-pyridyl)-5H-imidazolin-4-one

A mixture of 2,2′-pyridil (15.8 g, 74.4 mmol) and 2-guanidinobenzimidazole (19.5 g, 111.7 mmol) in methanol (440 mL) was treated with a solution of sodium hydroxide (2.97 g, 74.4 mmol) in water (74 mL) and the resulting mixture was left standing at room temperature for 4 days. A crystalline material was filtered and the mother liquor allowed to stand for 3 weeks. The precipitated solid was filtered and dried under vacuum to give the title compound (10.5 g, 36%) as its sodium salt. ¹H NMR (300 MHz, d₆-DMSO) δ 11.55 (br s, 1H), 10.05 (br s, 1H), 8.47 (m, 2H), 7.76 (m, 2H), 7.68 (m, 2H), 7.25 (m, 4H), 6.90 (m, 2H). Further slow concentration of the mother liquor gave a third solid, which was filtered and dried under vacuum to give the title compound (1.25 g, 5%) as a solid. ¹H NMR (300 MHz, d₆-DMSO) δ 11.8 (br s, 2H), 10.5 (br s, 1H), 8.64 (m, 2H), 7.89 (m, 2H), 7.53 (d, J=8.0 Hz, 2H), 7.44 (m, 4H), 7.07 (m, 2H).

b) 1,4-bis[2-(2-benzimidazolylamino)-5,5-di(2-pyridyl)-4-oxo-4H-imidazolin-3-ylmethyl]benzene bis-trifluoroacetic Acid Salt

To a solution of 2-(2-benzimidazolylamino)-5,5-di(2-pyridyl)-5H-imidazolin-4-one (390 mg, 1.00 mmol) in DMF (1 mL) at room temperature was added α,α′-dibromo-p-xylene (120 mg, 0.45 mmol) in one portion. The reaction was stirred at room temperature for 12 hours then concentrated under reduced pressure. The residue was taken up in DMSO (5 mL ) and purified by reverse phase HPLC [ODS, 0-90% CH₃CN/H₂O (0.1% TFA)] to give the title compound as a yellow solid (240 mg, 50%) MS (ES+) m/e 841 [M+H]⁺.

### Example 20

### 2,6-bis[2-(2-benzimidazolylamino)-5,5-di(2-pyridyl)-4-oxo-5H-imidazolin-3-ylmethyl]pyidine bis-trifluoroacetic Acid Salt

Following the procedure of Example 19(a)-(b), except substituting 2,6-bis(bromomethyl)pyridine for α,α′dibromo-p-xylene, the title compound was prepared (2% overall). MS (ES+) m/e 842 [M+H]⁺.

### Example 21

### Preparation of 1,4-bis{[1-(2-benzimidazolyl)-1-guanidino]methyl}benzene

To a solution of 2-guanidinobenzimidazole (350 mg, 2.0 mmol) in DMF at 0° C. was added NaH (88 mg of a 60% dispersion in mineral oil, 2.2 mmol) in portions over five minutes. The solution was warmed to room temperature and allowed to stir for 45 minutes. The solution was cooled to 0° C. and α,α′-dibromo-p-xylene (264 mg, 1.0 mmol) was added in portions over 1 hour. The solution was stirred an additional hour, concentrated under reduced pressure, and taken up in ethyl acetate. The organic solution was washed with aqueous NH₄Cl, NaCl, dried over anhydrous Na₂SO₄, and concentrated under reduced pressure. The residue was purified by flash chromatography (silica gel, ethyl acetate) to give the title compound as a white powder (350 mg, 77%). MS (ES+) m/e 453 [M+H]⁺.

All publications including, but not limited to, patents and patent applications, cited in this specification, are herein incorporated by reference as if each individual publication were specifically and individually indicated to be incorporated by reference herein as though fully set forth.

The above description fully discloses the invention, including preferred embodiments thereof. Modifications and improvements of the embodiments specifically disclosed herein are within the scope of the following claims. Without further elaboration, one skilled in the art can, using the preceding description, utilize the present invention to its fullest extent. Therefore, the examples provided herein are to be construed as merely illustrative and are not a limitation of the scope of the present invention in any way. The embodiments of the invention in which an exclusive property or privilege is claimed are defined as follows.

What is claimed is:

1. A method for inducing chemotaxis in endothelial cells comprising contacting the cells with a chemotaxis-inducing-effective amount of stromal cell derived factor-1α (SDF 1-α) (SEQ ID NO:4) in combination with a carrier.

2. A method for stimulating angiogenesis in the vasculature of a patient in need thereof comprising contacting the vasculature with an angiogenesis-stimulating-effective amount of SDF 1-α, (SEQ ID NO:4) in combination with a carrier.

3. A method for stimulating angiogenesis in the vasculature of a patient comprising contacting the vasculature with an angiogenesis-stimulating-effective amount of an agonist of the interaction between CXCR4 (SEQ ID NO:2) and SDF 1-α (SEQ ID NO:4).

4. The method as claimed in claim 3, wherein the patient is suffering from a disease selected from the group consisting of: atherosclerosis, restenosis, ischaemic stroke, and spinal cord injury.

5. A method for inhibiting angiogenesis in the vasculature of a patient comprising contacting the vasculature with an angiogenesis-inhibiting-effective amount of an antagonist of the interaction between CXCR4 (SEQ ID NO:2) and SDF 1-α (SEQ ID NO:4).

6. The method as claimed in claim 5, wherein the patient is suffering from a disease or disorder selected from the group consisting of: viral, bacterial, fungal and protozoan infections, pain, cancer, diabetes, obesity, anorexia, bulimia, asthma, allergies, Parkinson's disease, acute heart failure, hypotension, hypertension, urinary retention, osteoporosis, angina pectoris, myocardial infarction, stroke, ulcers, benign prostatic hypertrophy, migraine, vomiting, psychotic and neurological disorders and dyskinesias, inflammatory diseases, such as rheumatoid arthritis, diabetic retinopathy, inflammatory bowel disease, atherosclerosis, restenosis, stroke, Alzheimer's disease, congestive heart failure, and cardiac remodeling; angiogenic diseases, such as solid tumors, Kaposi Sarcoma, rheumatoid arthritis, and diabetic retinopathy.


SEQUENCE LISTING (6 sequences)

SEQ ID NO: 1 - DNA, 1902 bp, Human
  unsure (158)(223)(225)(226)(335)(466)(520)(530)(645)(763)(825)(1120),(1183)
gtttgttggctgcggcagcaggtagcaaagtgacgccgagggcctgagtgctccagtagccaccgcatctggagaaccagcggttaccatggaggggatcmgagtatatacacttcagataactacaccgaggaaatgggctcaggggactasytsdnytmgsgdytgactccatgaaggaaccctgtttccgtgaagaaaatgctaatttcaatadsmkcrnannaaatcttcctgcccaccatctactccatcatcttcttaactggcattgtgktystgvggcaatggattggtcatcctggtcatgggttaccagaagaaactgagaaggngvvmgykkrscatgacggacaagtacaggctgcacctgtcagtggccgacctcctctttgmtdkyrhsvadtcatcacgcttcccttctgggcagttgatgccgtggcaaactggtactttvtwavdavanwygggaacttcctatgcaaggcagtccatgtcatctacacagtcaacctctagnckavhvytvnycagcagtgtcctcatcctggccttcatcagtctggaccgctacctggccassvasdryatcgtccacgccaccaacagtcagaggccaaggaagctgttggctgaaaagvhatnsrrkakgtggtctatgttggcgtctggatccctgccctcctgctgactattcccgavvyvgvwatdcttcatctttgccaacgtcagtgaggcagatgacagatatatctgtgaccanvsaddrycdgcttctaccccaatgacttgtgggtggttgtgttccagtttcagcacatcryndwvvvhatggttggccttatcctgcctggtattgtcatcctgtcctgctattgcatmvggvscyctatcatctccaagctgtcacactccaagggccaccagaagcgcaaggcccskshskghkrkatcaagaccacagtcatcctcatcctggctttcttcgcctgttggctgcctkttvaacwtactacattgggatcagcatcgactccttcatcctcctggaaatcatcaayygsdskgcaagggtgtgagtttgagaacactgtgcacaagtggatttccatcaccggcntvhkwstaggccctagctttcttccactgttgtctgaaccccatcctctatgctttcaahccnyacttggagccaaatttaaaacctctgcccagcacgcactcacctctgtgaggakktsahatsvscagagggtccagcctcaagatcctctccaaaggaaagcgaggtggacattrgsskskgkrgghcatctgtttccactgagtctgagtcttcaagttttcactccagctaacacssvstsssshssagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttacttagtgttatg

SEQ ID NO: 2 - protein, 352 aa, Human
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - DNA, 1847 bp, Human
tctccgtcagccgcattgcccgctcggcgtccggcccccgacccgtgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgcagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctggggcgccgccactgccttcacctcctctttcaaacctcagtgattggctctgtgggctccatgtagaagccactattactgggactgtctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgcttgagtgactgggtttgtgattgcctctgaagcctatgtatgccatggaggcactaacaaactctgaggtttccgaaatcagaagcgaaaaaatcagtgaataaaccatcatcttgccactaccccctcctgaagccacagcaggggttcaggttccaatcagaactgttggcaaggtgacatttccatgcatagatgcgatccacagaaggtcctggtggtatttgtaactttttgcaaggcatttttttatatatatttttgtgcacatttttttttacgattctttagaaaacaaatgtatttcaaaatatatttatagtcgaacaagtcatatatatgaatgagagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggta

SEQ ID NO: 4 - protein, 89 aa, Human
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 5 - DNA, 20 bp, Artificial Sequence
  Modified_base 491-510: Amplification primer for SEQ ID NO: 1
tayctsgcyatygtscaygc

SEQ ID NO: 6 - DNA, 20 bp, Artificial Sequence
  Modified_base 980-999: Amplification primer for SEQ ID NO: 1
aargcrtaratsaykggrtt